(19) United States
(12) Patent Application Publication
(10) Pub. No.: US 2004/0058873 A1
(43) Pub. Date: Mar. 25, 2004

(54) Method for treating or preventing Alzheimer's disease

(76) Inventors: Robert W. Esmond, Vienna, VA (US); Jack R. Wands, Waban, MA (US); Suzanne de la Monte, Greenwich, RI (US)

Correspondence Address: FINNEGAN, HENDERSON, FARABOW, GARRETT & DUNNER, LLP, 1300 I STREET, NW, WASHINGTON, DC, 20005, US

(21) Appl. No.: 10/669,281

(22) Filed: Sep. 23, 2003

Related U.S. Application Data
(63) Continuation of application No. 09/394,712, filed on Sep. 13, 1999.
Application No. 09/394,712 is a continuation of application No. PCT/US98/04731, filed on Mar. 12, 1998.

(51) Int. Cl. A61K 38/30; A61K 31/498; A61K 31/4439; A61K 31/426; A61K 31/28

(52) U.S. Cl. 514/12; 514/342; 514/369; 514/250; 514/492

(57) ABSTRACT

Disclosed is a method for treating or preventing Alzheimer's disease by restricting the level of metabolizable carbohydrate in the diet and/or administering to the patient an effective amount of an agent which reduces serum insulin levels.

### CROSS REFERENCE TO RELATED APPLICATIONS

The present application is a continuation of PCT/US98/04731 filed Mar. 12, 1998. The present application also claims the benefit of U.S. provisional application 60/039,607. The contents of each of these two applications are fully incorporated by reference herein.

### BACKGROUND OF THE INVENTION

1. Field of the Invention The present invention is in the field of medicinal chemistry. In particular, the present invention is related to a sunrising new method to treat or prevent Alzheimer's disease by dietary restriction of carbohydrates and/or administration of an agent which causes reduction in serum insulin levels.

2. Related Art

According to a recent review by Mairin B. Brennan published in Chemical and Engineering News 75(3):29-35 (1997), roughly 4 million people in the United States have Alzheimer's disease. Inherited or not, the disease manifests itself with progressively impaired memory leading to mental confusion as the disease systematically kills off nerve cells in the brain. (Brennan.)

The devastating consequences of Alzheimer's disease has led to a prodigious effort to identify drugs that might be useful for treating the condition. Two drugs are currently available for treating Alzheimer's symptoms. Cognex (tarcine), sold by Parke-Davis and CoCensys Inc. was approved by the FDA in 1993. Aricept, sold by Eisai of Japan, was approved late in 1996. Both drugs are designed to improve memory and cognition in the earlier stages of the disease. (Brennan.)

Alzheimer's disease is characterized by amyloid plaque that deposits around and between nerve cells in the brains. The plaques contain fibrillar aggregates of a small peptide called amyloid β-peptide. These plaques are centers for the degeneration of nerve endings. Whether the fibers themselves are themselves toxic is somewhat controversial, in view of transgenic animals which have been engineered to express amyloid β-peptide. These mice make amyloid deposits, and there is damage to nerve cells around the plaque, however, no further neuronal loss is seen in these mice. Thus, there appear to be other mechanisms involved. (Brennan.)

Whether the amyloid plaques are the cause or the consequence of the disease is a perplexing question according to Brennan. However, “all genetic routes to Alzheimer's known today, ‘act by increasing production or deposition of amyloid—or both,” quoting Dennis J. Selkoe, professor of neurology and neuroscience at Harvard Medical School. Laedtke, et al., Clinical Research 42(1):65A (1994), have also noted an epidemiological correlation between the deposition of anyloid in islet cells, leading to glucose intolerance and non-insulin-dependent diabetes mellitus, and amyloid β-protein deposition in brain cells, as associated with Alzheimer's disease. The authors conclude that there may be an overlap in the molecular defects that predispose to islet and brain amyloid, and therefore NIDDM and AD.

There is evidence of the over-expression of a protein called neural tread protein (NTP) in Alzheimer's disease neurons (see WO94/23756). This protein has been cloned (referred to as AD10-7), and expressed in cell-free culture.

The cathepsins are a family of enzymes that are usually located in lysosomes. It has been found that the inhibition of cathepsin D using an aspartyl protease inhibitor reduces the formation of β-amyloid protein and the resultant senile plaques. Thus inhibitors of cathepsin D, such as rhodanine derivatives, have been proposed as therapeutic agents for the treatment of Alzheimer's disease. See U.S. Pat. Nos. 5,716,975 and 5,523,314.

A number of companies are seeking new therapeutic agents which cross the blood-brain barrier and inhibit amyloid deposition. One such company is Athena Neurosciences, South San Francisco, who has engineered a transgenic mouse model for the disease. Athena is sorting through hundreds of molecules in a series to look for the best pharmaceutical to take into development. (Brennan.)

One drug candidate developed by Neo-Therapeutics, Irvine, Calif., is nearing clinical trials. The hypoxanthine analog (AIT-082) promotes nerve regeneration in the areas of the brain associated with memory. When the drug is administered directly to the brains of 13 month old mice, about 50% of the animals show a delay of about two months in any memory deficit and the other 50% never develop a memory deficit. This drug activates genes that express growth factor proteins known to reverse memory deficits in aged rodents when directly delivered to the brain. (Brennan.)

Another memory enhancing drug ready for clinical trials is CX516, codeveloped by Gary S. Lynch, a professor of psychobiology at the University of California, Irvine, and Gary A. Rogers, vice president of pharmaceutical discovery at Cirtex Pharmaceuticals, Irvine, Calif. CX516 is an agonist of the AMPA receptor, and promotes the uptake of Ca²⁺ into nerve cells when the brain levels of glutamate are low, as they are in Alzheimer's disease. This drug reversed age-associated memory impairment in rats. (Brennan.)

An over the counter agent that may lessen the symptoms or delay the progression of the disease is the nicotine patch. According to Ken Kellar, a professor of pharmacology at the Georgetown University Medical School, Washington, D.C., epidemiological data indicate that there is a lower incidence of Alzheimer's disease among people who smoke. The nicotine patch is now being tested in 12 month clinical study. (Brennan.)

Estrogen is also being evaluated as an agent that might be helpful in protecting women from Alzheimer's disease. Preliminary results indicate that women who receive estrogen replacement therapy have a lower risk of developing the disease. (Brennan.)

Another agent being evaluated is prednisone. This drug is being tested to see if it can benefit Alzheimer's patients by reducing inflammation in their brains. A further study has just been completed which examined the antioxidant effect of vitamin E and selegiline, a drug used to treat Parkinson's disease. (Brennan.)

In completely unrelated studies, it has been reported that elevated levels of insulin in the body are responsible for many cases of obesity, diabetes, heart disease, high blood pressure, and high cholesterol levels. Michael R. Eades and Mary Dan Eades, “Protein Power,” Bantam Books, New York, N.Y. (1996). Patients with any of these conditions have been successfully treated with a dietetic regimen which is designed to reduce insulin levels, primarily by strict limitation of metabolizable carbohydrate in the diet. A further strategy is to ameliorate insulin insensitivity which progresses in severity in middle age, by adding chromium to the diet. By reducing insulin insensitivity, lower levels of insulin are required by the body to clear glucose from the blood.

### SUMMARY OF THE INVENTION

The present invention is related to the discovery that high levels of circulating insulin are a root cause of Alzheimer's disease. In particular, it has been discovered that insulin stimulates the increased expression of NTP in nerve cell culture. Since insulin crosses the blood-brain barrier, it is now clear that high levels of insulin stimulate brain nerve cells to secrete NTP and develop the hallmarks of Alzheimer's disease.

The present invention is directed to the treatment or prevention of Alzheimer's disease, in a human, comprising administering to an animal in need thereof an effective amount of an agent which results in lowered serum insulin levels. The agent useful in the present invention is one that is also useful for treating impaired glucose tolerance.

The present invention is also directed to the treatment or prevention of Alzheimer's disease, in a human, comprising restricting the metabolizable carbohydrates in the diet of the human to a level which results in lowered serum insulin levels.

The present invention also relates to a method of improving mentation of a patient with Alzheimer's disease, comprising administering to said patient an effective amount of an agent which increases the insulin sensitivity of the patient.

The present invention also relates to a method of treating or preventing Alzheimer's disease, in a human, comprising administering to an animal in need thereof an effective amount of an agent which results in lowered serum insulin levels and an agent which inhibits the formation of small strokes.

### DETAILED DESCRIPTION OF THE PREFERRED EMBODIMENTS

Animals with insulin insensitivity require higher levels of serum insulin to stimulate the metabolism of serum glucose and storage for later use. Although insulin has countless other actions in the body, the main function of insulin is to prevent serum glucose levels from rising too high. Thus, when glucose levels rise, insulin levels rise. However, when cells become resistant to insulin, the insulin receptors begin to malfunction. This malfunction appears to be a result of inherited tendencies and lifestyle abuse (over-consumption of carbohydrates). Thus, the receptors require higher levels of insulin to allow the glucose to be removed from the blood. While low levels of insulin are necessary to clear serum glucose when the insulin receptors are working optimally, insulin insensitive receptors require an excess level of insulin to keep serum glucose within the normal range.

Insulin insensitivity can be diagnosed by determining whether the animal has an elevated insulin level. In the case of humans, insulin levels of over 10 mU/ml indicate that the person has at least some insulin insensitivity. Eades and Eades, supra. Insulin values of 25-50 or more are very high and indicative of a high level of insulin resistance. People with insulin levels above 10 mU/ml are considered to be in need of treatment to reduce insulin levels and thereby treat, prevent or reduce the possibility of having Alzheimer's disease in the future.

Agents which may be administered to animals which lower serum insulin levels include drugs which are known to be useful for treating insulin insensitivity. One example of such an agent is chromium. The insulin receptor requires chromium to function properly. Deficiency of chromium is rampant in the American population as a diet high in starch and sugar puts a heavy demand on the insulin system to handle the incoming carbohydrates. Thus, 100-300 micrograms per day of chromium supplements may be administered, e.g. orally or systemically. Preferably, the dose is 200 micrograms of chromium per day. Preferably, the chromium is administered in the form of a chelate. A preferred chromium chelate is niacin bound chromium.

Another agent which can be used is human insulin-like growth factor I (hIGF-I). Recombinant hIGF-I has been reported to be useful for reducing hyperglycemia in patients with extreme insulin resistance. Schoenle et al., Diabetologia 34:675-679 (1991). See also Usala et al., N. Engl. J Med 32 7:853-857 (1992); and Zenobi et al., J. Clin. Invest. 89:1908-1913 (1992). Thus, hIGF-I may be administered by intraperitoneal means to a human in need thereof to treat or prevent the onset of Alzheimer's disease. hIGF-I may be administered, e.g. systemically by injection, to the patient in need thereof in an amount effective which can be determined with no more than routine experimentation.

Other agents which can be used in the practice of the invention include dopamine agonists which have been reported to be useful for treating insulin resistance. See U.S. Pat. No. 5,468,755. An example of a dopamine agonist that can be used is bromocriptine. Other dopamine agonists are described in U.S. Pat. Nos. 5,597,832, 5,602,120 and 5,602,121. Thus, a dopamine agonist may be administered to a human in need thereof to treat or prevent the onset of Alzheimer's disease. Routes of administration for such dopamine agonists are described in U.S. Pat. Nos. 5,468,755, 5,597,832, 5,602,120 and 5,602,121. The dopamine agonist may be administered to the patient in need thereof in an amount effective which is, in general, the amount required for the dopamine agonist to treat insulin resistance according to U.S. Pat. No. 5,468,755.

Other agents which can be used in the practice of the invention include pyruvate and pyruvate precursors which have been reported to improve insulin resistance and lower fasting insulin levels. See U.S. Pat. Nos. 5,472,980 and 5,283,260.

Other agents which can be used in the practice of the invention include thiazolidinediones and related antihyperglycemic agents which have been reported to be useful for treating impaired glucose tolerance in order to prevent or delay the onset of non-insulin-dependent diabetes mellitus. See U.S. Pat. No. 5,478,852. An example of a thiazolidinedione that can be used is troglitazone (brand name Rezulin™) that has recently been approved by the U.S. Food and Drug Administration for treating insulin resistance. Routes of administration for such thiazolidinediones and related antihyperglycemic agents are described in U.S. Pat. No. 5,478,852. The thiazolidinediones and related antihyperglycemic agents may be administered to the patient in an amount effective which is, in general, the amount effect to treat impaired glucose tolerance according to U.S. Pat. No. 5,478,852. See also, U.S. Pat. No. 5,457,109. Unlike sulfonylureas, troglitazone is not an insulin secretagogue, “Physicians' Desk Reference,” Medical Economics Company, Montvale, N.J., 2118-2119 (1998).

Additional antihyperglycemic agents include, inter alia, rhodanine derivatives such as the 5-methylene-2-thioxo-4-thiazolidinones, see U.S. Pat. No. 5,716,975; C-substituted pentacycloazoles and N-alkyl-substituted pentacycloazoles, see U.S. Pat. No. 5,641,796; hydroxyurea derivatives, see U.S. Pat. Nos. 5,646,168 and 5,463,070; and piperazinylalkylpyrimidines, see U.S. Pat. No. 4,980,350.

Other agents which can be used in the practice of the invention include benzothiodiazines and related antihypoglycemic agents which have been reported to be useful for treating symptomatic hypoglycemia. These agents function by suppressing insulin levels, thereby causing an increased glucose level in the blood. An example of a benzothiadiazine which can be used is diazoxide (brand name Proglycem™) which is approved by the U.S. Food and Drug Administration for treating hypoglycemia due to hyperinsulinism. See, “Physicians' Desk Reference,” Medical Economics Company, Montvale, N.J., 595-597 (1998).

A second method of the invention is directed to the treatment or prevention of Alzheimer's disease by the restriction of metabolizable carbohydrate in the diet. According to the invention, the amount of metabolizable carbohydrate is considered restricted if no more than about 55 grams are ingested per day. Preferably, no more than about 30 grams of metabolizable carbohydrates are ingested. More preferably, no more than about 15 grams of metabolizable cabohydrates are ingested. Most preferably, no more than about 10 grams of metabolizable carbohydrates are ingested. One can easily achieve these lowered levels of carbohydrate ingestion by following the regimens disclosed by Michael R. Eades and Mary Dan Eades in their book entitled “Protein Power,” Bantam Books, New York, N.Y. (1996). The regimen disclosed by Michael R. Eades and Mary Dan Eades is designed to reduce serum insulin levels to normal levels and, thereby, treat the symptoms of insulin insensitivity including obesity, diabetes, heart disease, high blood pressure and high cholesterol and triglyceride levels.

Further, one can easily adjust the levels of carbohydrates in the diet by reading nutrition labels on foods. The carbohydrate level on food labels includes the non-metabolizable fiber content. Thus, when determining the metabolizable carbohydrate amount in a serving of the food, the number of grams of fiber must be subtracted. In general, to achieve a diet which is low in metabolizable carbohydrates, one must ingest large amounts of protein from red meat, fowl and fish; vegetables including green leafy vegetables, tomatoes, peppers, avocados, broccoli, egg-plant, zucchini, green beans, asparagus, celery, cucumber, mushrooms and salads. Michael R. Eades and Mary Dan Eades disclose the amounts of metabolizable carbohydrates in a large number of foods which allows one to plan a diet that is very low in metabolizable carbohydrates. See also Robert C. Atkins and Veronica Atkins, “Dr. Atkin's Quick and Easy New Diet Cookbook,” Fireside Books, New York, N.Y. (1997).

The present invention also relates to a method of improving mentation of a patient with Alzheimer's disease, comprising administering to said patient an effective amount of an agent which increases the insulin sensitivity of the patient. Several lines of investigation suggest a link between impaired glucose utilization and Alzheimer's disease. This hypothesis has been supported by findings that raising plasma glucose levels through glucose administration in elderly humans and rodents improves memory without affecting motor and nonmemory functions. Craft, S., et al., “Effects of Hyperglycemia on Memory and Hormone Levels in Dementia of the Alzheimer Type: A Longitudinal Study,” Behav. Neurosci. 107:926-940 (1993). Thus, according to the present invention, an agent may be administered to a patient with Alzheimer's disease to improve mentation, which agent is effective for treating insulin insensitivity. By decreasing insulin insensitivity, that is by increasing insulin sensitivity, in the patient, glucose utilization is improved in the brain and mentation will improve.

Agents which inhibit the formation of small strokes include aspirin.

The agents described herein may also be administered in conjunction with an antiinflammatory agent such as ibuprofen which has been found useful in some studies in ameliorating Alzheimer's disease.

The agents that have been described herein may also be administered with compounds which modulate ATP production and have thereby been found useful as an alternative energy source to glucose for conditions in which ischemic or hypoxic conditions have compromised ATP production. Such compounds include, inter alia, fructose-1,6-biphosphate, see U.S. Pat. Nos. 4,546,095, 4,703,040, 4,757,052, and 5,039,665; pyruvate, see U.S. Pat. No. 5,395,822; glyceraldehyde-3-phosphate and 3-phosphoglycerate, see U.S. Pat. No. 5,707,971. Administration of these agents may also be useful as an alternative to insulin treatment by providing an energy source alternative to glucose, and may obviate the general decline of aging by enhancing ATP production according to U.S. Pat. No. 5,707,971.

Having now generally described the invention, the same will be more readily understood through reference to the following Examples which are provided by way of illustration, and are not intended to be limiting of the present invention, unless specified.

### EXAMPLES

### Example 1

Insulin Stimulates the Expression of AD7c-NTP, a Protein Which Causes Neurons to Exhibit Neuronal Sprouting and Apoptosis

Insulin is an important mediator of growth and differentiation in CNS neurons. Insulin stimulated differentiation of PNET2 cells was associated with rapid (within 10 minutes) but transient increases in the levels of the 39 kD, 18 kD and 15 kD NTP species, followed by sustained increases in synthesis and steady state levels of all five NTP species. In contrast, the failure of insulin to induce differentiation of PNET1 cells was associated with absent insulin modulation of NTP.

Analysis of the signal transduction pathways demonstrated that the insulin-induced up-regulation of NTP molecules in PNET2 cells was mediated through phosphorylation of the insulin receptor substrate-1 (IRS-1) and the insulin receptor β subunit (IRβs) itself. In PNET1 cells, the lack of insulin responsiveness was associated with impaired insulin-mediated tyrosyl phosphorylation of IRS-1, but normal insulin receptor phosphorylation. Correspondingly, the insulin-stimulated association between P13 kinase and phosphorylated IRS-1 was also impaired in PNET1 cells. In essence, impaired insulin-mediated tyrosyl phosphorylation of IRS-1 in PNET1 cells halted activation of the insulin signal transduction cascade, and subsequent events leading to modulated gene (NTP) expression. PNET1 cells lacked insulin responsiveness and failed to phosphorylate IRS-1, but insulin receptor levels and tyrosyl phosphorylation (PY) of the β-subunit were intact. PNET2 cells responded to insulin stimulation with phosphorylation of IRS-1, up-regulation of NTP, and neuronal differentiation. The results were confirmed by absent association between PI3 kinase and IRS-1-PY in PNET1 cells after insulin stimulation.

Neuritic sprouting and neuronal differentiation were induced in PNET2 and SH-Sy5y cells by insulin, PMA, or RA stimulation. Insulin-mediated neuritic growth was associated with increased expression of the fetal brain and PNET-dominant forms of NTP (15 kD and 18 kD). In contrast, the PMA- and RA-induced neuritic sprouting modulated expression of the 21 kD and 26 kD NTP species, which are primarily expressed in the mature brain, and accumulated in AD brains. Thus, expression of the immature or fetal forms of NTP are regulated by mechanisms and growth factors distinct from those involved in modulating expression of the 21 kD and 26 kD NTP molecules. Therefore, expression of fetal NTP molecules/genes can be mediated through the IRS-1 cascade, whereas expression of adult brain/AD-associated NTP genes can be regulated mainly through protein kinase C pathways.

From the foregoing description, one skilled in the art can easily ascertain the essential characteristics of this invention, and without departing from the spirit and scope thereof, can make various changes and modifications of the invention to adapt it to various usages and conditions without undue experimentation. All patents, patent applications and publications cited herein are incorporated by reference in their entirety.

What is claimed is:

1. A method for the treatment or prevention of Alzheimer's disease, in a human, comprising administering to a human in need thereof an effective amount of an agent which results in lowered serum insulin levels.

2. The method of claim 1, wherein said agent is chromium.

3. The method of claim 1, wherein said agent is insulin-like growth factor.

4. The method of claim 1, wherein said agent is a dopamine agonist.

5. The method of claim 4, wherein said dopamine agonist is bromocryptine.

6. The method of claim 1, wherein said agent is a thiazolidinedione.

7. The method of claim 6, wherein said thiazolidinedione is troglitazone.

8. A method for the treatment or prevention of Alzheimer's disease, in a human, comprising restricting the metabolizable carbohydrates in the diet of the human to a level which results in lowered serum insulin levels.

9. The method of claim 8, wherein the metabolizable carbohydrates in the diet are limited to no more than about 55 grams per day.

10. The method of claim 8, wherein the metabolizable carbohydrates in the diet are limited to no more than about 30 grams per day.

11. The method of claim 8, wherein the metabolizable carbohydrates in the diet are limited to no more than about 15 grams per day.

12. The method of claim 8, wherein the metabolizable carbohydrates in the diet are limited to no more than about 10 grams per day.

13. A method for the treatment or prevention of Alzheimer's disease, in a human, comprising administering to a human in need thereof an effective amount of an agent which results in lowered serum insulin levels and restricting the metabolizable carbohydrates in the diet of the human.

14. The method of claim 13, wherein said agent is selected from the group consisting of chromium, insulin-like growth factor, a dopamine agonist and a thiazolidinedione.

15. The method of claim 13, wherein said agent is troglitazone.

16. The method of claim 13, wherein the metabolizable carbohydrates in the diet are limited to no more than about 55 grams per day.

17. The method of claim 13, wherein the metabolizable carbohydrates in the diet are limited to no more than about 30 grams per day.

18. The method of claim 13, wherein the metabolizable carbohydrates in the diet are limited to no more than about 15 grams per day.

19. The method of claim 13, wherein the metabolizable carbohydrates in the diet are limited to no more than about 10 grams per day.

20. A method of improving mentation of a patient with Alzheimer's disease, comprising administering to said patient an effective amount of an agent which increases the insulin sensitivity of the patient.
